(19) United States
(12) Patent Application Publication
(10) Pub. No.: US 2009/0082722 A1
(43) Pub. Date: Mar. 26, 2009

(54) REMOTE NAVIGATION ADVANCER DEVICES AND METHODS OF USE

(76) Inventors: Gareth T. Munger, St. Louis, MO (US); Janet Adair, St. Louis, MO (US); Ashwini K. Pandey, Marlborough, MA (US); John B. Kinder, JR., St. Louis, MO (US); John C. Knudson, Edina, MN (US); Christopher D. Minar, New Prague, MN (US); Roger G. Riedel, JR., Mahtomedi, MN (US); Steven E. Scott, Chanhassen, MN (US); Scott Klimek, Maple Grove, MN (US); Alan D. Eskuri, Hanover, MN (US); Amy R. Raatikka, Plymouth, MN (US); Pete Skujins, Minneapolis, MN (US); Stephen W. Pryor, Forest Lake, MN (US)

Correspondence Address: Bryan K. Wheelock;Suite 400, 7700 Bonhomme, St. Louis, MO, 63105, US

(21) Appl. No.: 12/196,214

(22) Filed: Aug. 21, 2008

Related U.S. Application Data
(60) Provisional application No. 60/957,008, filed on Aug. 21, 2007.

(51) Int. Cl. A61M 25/092

(52) U.S. Cl. 604/95.01

(57) ABSTRACT

Various systems for advancing medical devices within a subject's body are provided that configured to engage the outer surface of the medical device at a number of points about the circumference of the medical device, to thereby grip the medical device for enabling advancement of the device. The advancing systems include engaging portions configured to engage the outer surface of a medical device at a number of points about the circumference of the medical device. The engaging portions preferably engage opposing sides of the medical device's outer surface over a predetermined longitudinal length, to clamp against a longitudinal portion of the medical device that provides improved surface contact with the medical device over conventional roller guides. Various methods for performing medical procedures utilizing various medical devices and advancer systems are also provided.

### CROSS-REFERENCE TO RELATED APPLICATION

This application claims priority to prior U.S. Provisional Application Ser. No. 60/957,008, filed Aug. 21, 2007, the entire disclosure of which is incorporated herein by reference.

### FIELD OF THE INVENTION

This invention relates to devices for the control of multiple interventional devices, and methods of using the same. The controls enable advancement, retraction, rotation, and deflection of multiple devices. In particular, the controls enable simultaneous motion of a multiplicity of devices with respect to one another, along several degrees of freedom including linear motion, rotation, and deflection. Methods of using such control in the context of minimally interventional procedures are described. In one particular embodiment, such methods include simultaneous control of an image device and an ablation device.

### BACKGROUND OF THE INVENTION

Minimally invasive intervention systems include navigation systems, such as the Niobe™ magnetic navigation system developed by Stereotaxis, St. Louis, Mo. Such systems typically comprise an imaging means for real-time guidance and monitoring of the intervention; additional feedback is provided by a three-dimensional (3D) localization system that allows real time determination of the catheter or interventional device tip position and orientation with respect to the operating room and, through co-registered imaging, with respect to the patient.

The availability of methods and systems for safe, efficient minimally invasive interventions have greatly impacted and changed the practice of cardiac and vascular treatment delivery in the last decade. The treatment of a number of cardiac disorders has become possible without requiring open heart surgery. In particular, progress in vascular interventions such as crossing and opening of occluded and stenosed arteries, placement of stents, and local delivery of therapeutic agents have significantly helped in reducing the morbidity and mortality related to coronary arteries impairment and associated cardiac ischemia.

As methods and technologies evolve, treatment is considered for smaller and narrower arteries in more complex anatomy. Situations up-to-now considered outside the realm of minimally invasive techniques are now being evaluated for intervention with new devices and methods. Difficult cases, such as the treatment of chronic totally occluded (CTO) lesions, are still not practical due to the increased risk of adverse events when the lesions cannot be properly imaged, the distal vessel not easily accessible, or dense fibrous lesions prevent appropriate visualization of the vessel walls.

### SUMMARY OF THE INVENTION

The present invention relates to devices for the simultaneous control of a plurality of interventional devices, and methods of using such devices for the successful treatment of complex cases up-to-now out of the reach of minimally invasive interventional systems.

More specifically this invention relates to the automatic, remote control of a plurality of devices within the vasculature or hollow organs of a subject. In a specific embodiment, the present invention describes the simultaneous control and progression of an imaging device and of a treatment device; in such an embodiment, an ablation device creates a small incremental lumen in a lesion, and the imaging device is advanced correspondingly to maintain the ablation device in the field of view and to enable visualization of the respective positions of the ablation device and the organ walls.

Further areas of applicability of the present invention will become apparent from the detailed description provided hereinafter. It should be understood that the detailed description and specific examples, while indicating the preferred embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1-A is a schematic diagram showing a patient positioned in a projection imaging and interventional system for a minimally invasive procedure such as a coronary arteries diagnostic and therapeutic intervention;

FIG. 1-B schematically illustrates an interventional device distal end being navigated through one of the patient's vessels in the vicinity of an implant such as an arterial stent;

FIG. 2 presents a functional block diagram of a preferred embodiment of the present invention as applied to a therapeutic treatment;

FIG. 3 presents a functional block diagram of an alternate preferred embodiment applied to therapeutic treatment;

FIG. 4 shows an embodiment of a shaped-wire inside of a magnetic Electrophysiology Catheter.

FIG. 5A shows an embodiment of a pre-bent wire-guide having a pre-determined bend angle near the distal end portion.

FIG. 5B shows an alternate configuration of a pre-bent J-shaped wire-guide with pacing electrodes capable of sensing electrical activity.

FIG. 6 illustrates advancer movement of the J-shaped wire-guide shown in FIG. 5.

FIG. 7 shows an embodiment of an ElectroPhysiology catheter disposed within a sheath.

FIG. 8 shows an embodiment of an RF wire device disposed within a wire-guide.

FIG. 9 shows another embodiment of an RF wire device disposed within a wire-guide having multiple lumens.

FIG. 10 shows an embodiment of a Catheter Advancing System for a core guide-wire, having a reel on which the core guide-wire is coiled.

FIG. 11 shows an embodiment of a core guide-wire drive mechanism with a reel on an axle.

FIG. 12 shows core guide-wire drive mechanism of FIG. 11, with the core guide-wire fed through a plurality of guides.

FIG. 13 shows the core guide-wire drive mechanism of FIG. 11.

FIG. 14 shows one embodiment of a portion of an advancer apparatus.

FIG. 15 shows the advancer apparatus of FIG. 14 in an open position.

FIG. 16 shows another embodiment of an advancer mechanism.

FIG. 17 shows another embodiment of an advancer mechanism, in both an unclamped position and a clamped position around a medical device.

FIG. 18 shows another embodiment of an advancer mechanism.

FIG. 19 shows a cross-section of a portion of the advancer embodiment in FIG. 18.

FIG. 20 shows a cross-section of the advancer embodiment in FIG. 18.

FIG. 21 shows an alternate cross-section of a portion of the advancer embodiment in FIG. 18.

FIG. 22 shows another embodiment of an advancer mechanism.

FIG. 23 shows portions of the advancer embodiment in FIG. 22.

FIG. 24 shows portions of the advancer embodiment in FIG. 22.

FIG. 25 shows another embodiment of an advancer mechanism.

FIG. 26 shows another embodiment of an advancer mechanism.

FIG. 27 shows another embodiment of an advancer mechanism.

FIG. 28 shows another embodiment of an advancer mechanism.

FIG. 29 shows another embodiment of an advancer mechanism.

FIG. 30 shows portions of the advancer embodiment in FIG. 29.

FIG. 31 shows portions of the advancer embodiment in FIG. 29.

FIG. 32 shows portions of the advancer embodiment in FIG. 29.

FIG. 33 shows portions of the advancer embodiment in FIG. 29.

FIG. 34 shows another embodiment of an advancer mechanism.

FIG. 35 illustrates the wave propagation advancer in FIG. 34.

FIG. 36 shows another embodiment of an advancer mechanism.

FIG. 37 shows another embodiment of an advancer mechanism.

FIG. 38 shows another embodiment of an advancer mechanism.

FIG. 39 shows another embodiment of an advancer mechanism.

FIG. 40 shows another embodiment of an advancer mechanism.

FIG. 41 illustrates an application of one embodiment of an advancer mechanism, for advancing catheters into a subject's body.

Corresponding reference numerals indicate corresponding parts throughout the several views of the drawings.

### DETAILED DESCRIPTION OF THE INVENTION

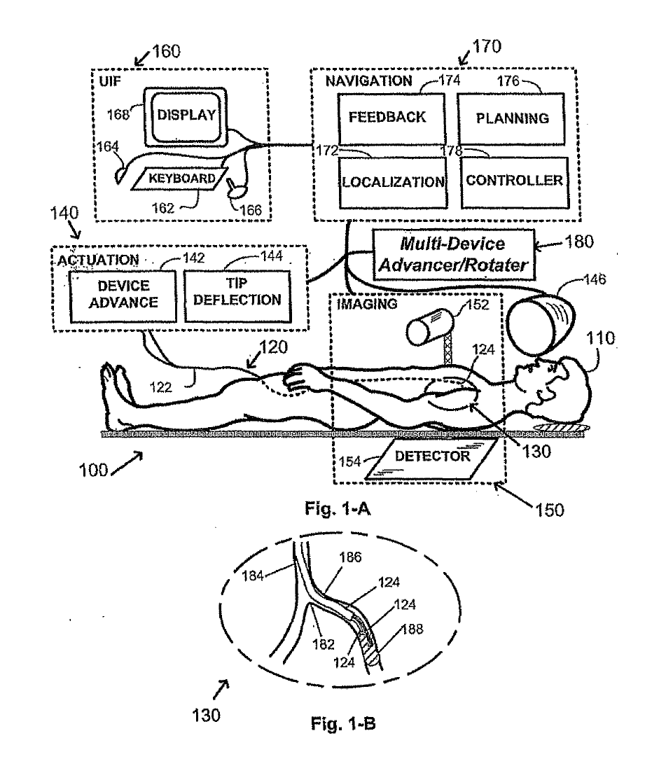

As illustrated in FIG. 1-A, a patient 110 is positioned within a remotely actuated, computer controlled interventional system 100. An elongated navigable medical device 120 having a proximal end 122 and a distal end 124 is provided for use in the interventional system 100 and the medical device is inserted into a blood vessel of the patient and navigated to an intervention volume 130. A means of applying force or torque to advance or orient the device distal end 124 is provided, as illustrated by actuation block 140 comprising a component 142 capable of precise proximal device advance and retraction and a tip deflection component 144. The actuation sub-system for tip deflection may be one of (i) a mechanical pull-wire system; (ii) a hydraulic or pneumatic system; (iii) an electrostrictive system; (iv) a magnetostrictive system; (v) a magnetic system; or (vi) other navigation system as known in the art. For illustration of a preferred embodiment, in magnetic navigation a magnetic field externally generated by magnets(s) assembly 146 orients a small magnetically responsive element (not shown) located at or near the device distal end 124. Real time information is provided to the physician by an imaging sub-system 150, for example an x-ray imaging chain comprising an x-ray tube 152 and a digital x-ray detector 154, to facilitate planning and guidance of the procedure. Additional real-time information such as distal tip position and orientation may be supplied by use of a three-dimensional (3D) device localization sub-system such as comprising a set of electromagnetic wave receivers located at the device distal end (not shown), and associated external electromagnetic wave emitters (not shown); or other localization device with similar effect such as an electric field-based localization system that measures local fields induced by an externally applied voltage gradient. In the latter case the conducting body of a wire within the device itself carries the signal recorded by the tip electrode to a proximally located localization system. The physician provides inputs to the navigation system through a user interface (UIF) sub-system 160 comprising user interfaces devices such as keyboard 162, mouse 164, joystick 166, display 168, and similar input or output devices. Display 168 also shows real-time image information acquired by the imaging system 150 and localization information acquired by the three-dimensional localization system. UIF sub-system 160 relays inputs from the user to a navigation sub-system 170 comprising 3D localization block 172, feedback block 174, planning block 176, and controller 178. Navigation control sequences are determined by the planning block 176 based on inputs from the user, and also possibly determined from pre-operative or intra-operative image data and localization data from a localization device and sub-system as described above and processed by localization block 172, and alternatively or additionally real-time imaging or additional feedback data processed by feedback block 174. The navigation control sequence instructions are then sent to controller 178 that actuates interventional device 120 through actuation block 140 to effect device advance or retraction and tip deflection. Other navigation sensors might include an ultrasound device or other device appropriate for the determination of distances from the device tip to surrounding tissues, or for tissue characterization. Further device tip feedback data may include relative tip and tissues positions information provided by a local intra-operative imaging system, and predictive device modeling and representation. Such device feedback in particular enables remote control of the intervention. In closed-loop implementations, the navigation sub-system 170 automatically provides input commands to the device advance/retraction 142 and tip orientation 144 actuation components based on feedback data and previously provided input instructions; in semi closed-loop implementations, the physician fine-tunes the navigation control, based in part upon displayed information and possibly other feedback data, such as haptic force feedback. Control commands and feedback data may be communicated from the user interface 160 and navigation sub-system 170 to the device and from the device back to navigation sub-system 170 and the user through cables or other means, such as wireless communications and interfaces. Additionally, FIG. 1-A schematically shows magnetic cell delivery block 180 that performs specific functions in various embodiments of the present invention. Cell delivery block 180 applies to magnetic navigation system such as that illustrated in FIG. 1-A, and more generally to any medical navigation device that also comprises an external magnet for the generation of specific magnetic field sequences during cell delivery, as described in this disclosure.

FIG. 1-B schematically shows the distal end 124 of a master interventional device 120 having progressed through a branch 182 of the coronary arterial tree 184 into the left branch 186 and toward a chronic total occlusion 188. The interventional device 120 is provided either with a multiplicity of independent lumen, or with a hollow lumen capable of supporting simultaneous and independent progression of several interventional devices. In FIG. 1-B, an imaging or sensing device, 192, such as an optical imaging catheter is advanced past the distal end of master device 120 and to the vicinity of CTO 188. Simultaneously or subsequently, an ablative device 194, such as an RF wire is navigated to make contact with the CTO cap and ablate under imaging control a small incremental path through the lesion. After such incremental ablation, the RF wire is retracted, and the imaging or sensing device is advanced through the newly created lesion partial lumen to assess the respective position of the ablation path with respect to the organ wall. Adjustments to the RF wire control and navigation path are made based upon this assessment, the imaging or sensing catheter is retracted, and the intervention resume by iteratively applying ablative power, imaging or sensing, until the CTO has been successfully and safely crossed.

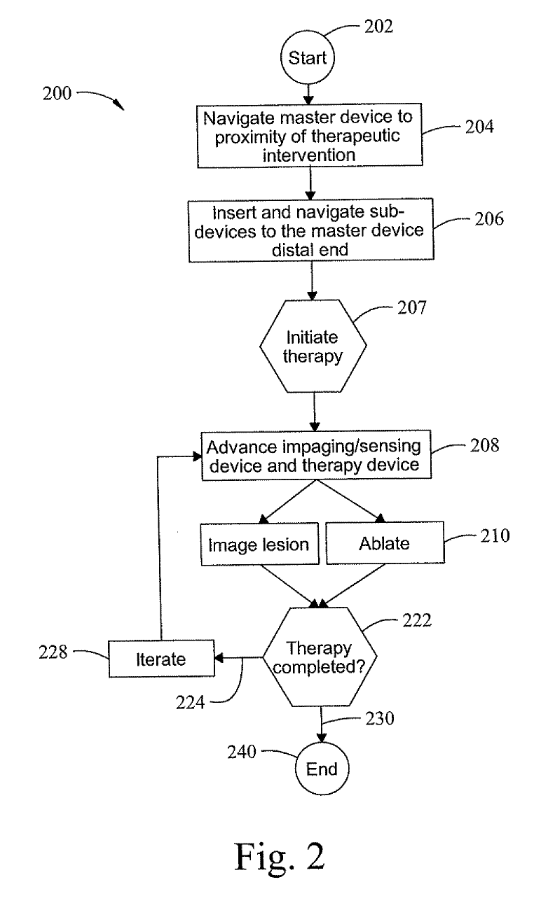

FIG. 2 presents a flowchart 200 for the steps of an intervention according to the previous description. Following intervention start, 202, a master interventional device is navigated to the proximity of the lesion or area to be treated, 204. Areas to be treated include, for example, cardiac wall chamber tissues presenting foci of spurious electrical activity; narrowed or stenosed arteries, such as the coronary arteries. Then in step 206, sub-devices for the characterization of tissue and the treatment of areas or lesions are inserted proximally into the master device and advanced through its distal end to the vicinity of the region of interest. Examples include the advancement of an ablative device for electrophysiology cardiac chamber treatment, or the advancement of a device for the crossing of an occlusion, such as a CTO. Imaging or characterizing devices include optical fiber optic device for optical coherence tomography (OCT), optical reflectometry, intra-vascular ultrasound (IVUS) devices, and others as known in the art. After therapy initiation, step 207, both the imaging/characterizing and the therapy devices are advanced to the lesion or area to be treated, either simultaneously or in sequence, step 208. With the ablation device in the field of view of the imaging or sensing device, ablation is initiated, 210, under monitoring, guidance and control of the real-time imaging/sensing device, 212. At regular intervals, either predetermined or determined upon real-time intervention parameters, or under physician guidance, the results of the intervention are evaluated, 222. Should the therapy be completed, step 230, the method terminates at 240. Otherwise, the method iterates through steps 224, 228, the imaging/sensing and treatment devices are advanced as necessary for safe and effective treatment, and the intervention resumes till satisfactory results have been achieved.

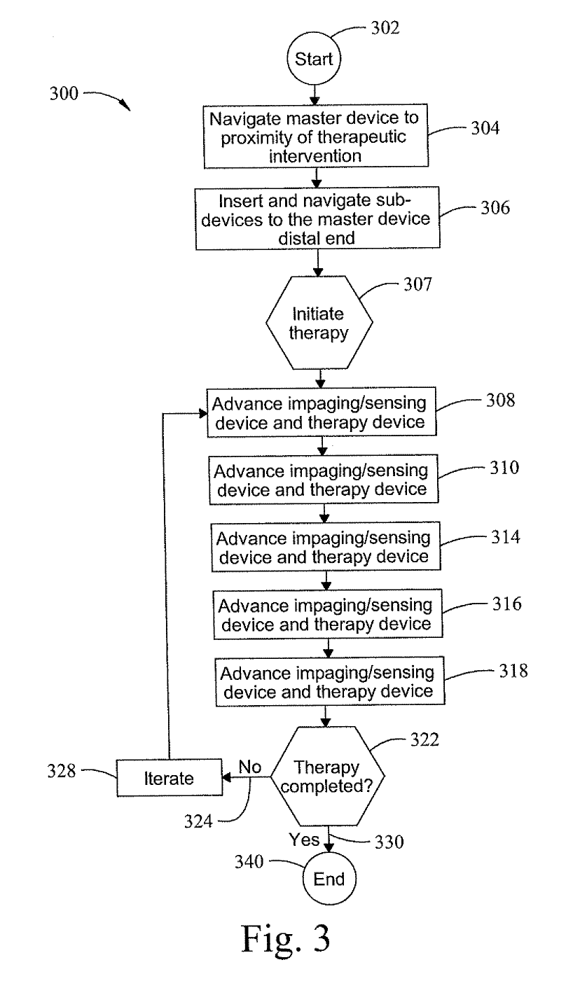

In an alternative embodiment of the present invention, the steps of imaging/sensing and ablating are sequential rather than simultaneous. FIG. 3 illustrates the work flow for such an embodiment. As above the master device is navigated to the theater of intervention, step 304, and sub-devices are inserted within and navigated through the master device distal end, step 305. There, upon therapy initiation, 307 the imaging/sensing device is advanced to the vicinity of the treatment area, 308, acquires data sufficient for the navigation and guidance of the treatment, 310, and the ablation or treatment device itself is subsequently navigated and actuated 314 to enable partial therapy completion. Following either application of a pre-determined power amount, or progression and advance over a predetermined distance through the lesion, or combination thereof, power application to the ablating devices stops, the device is retracted if necessary to allow the imaging/sensing device to progress to the most recently treated area, step 316. The parameters of the treatment, and the navigation sequence steps, are adjusted in step 318 as necessary to enable safe and effective lesion treatment. As above, the method iterates as necessary through steps 324 and 328 to complete the therapy, 330.

In the two workflows illustrated in FIGS. 2 and 3, the term “advance” is meant to encompass device length advance and retraction, rotation, and deflection, as appropriate for the fine control of the interventional device. Fine device control is enabled by a sub-system such as a “catheter advance system” (CAS) that permits individual and simultaneous control of several interventional devices, under either computer or user command. Device control includes device linear length advance or retraction, device rotation, and for a least a subset of devices, device distal end deflection control. This later type of motion is enacted for example by applying a set of pull-wire tensions on a set of wires, each pull wire being provided with an independent tension controller. Additionally or alternatively, such pull wire tensions may be transmitted to an intermediate point or set of points along the device; such device locations possibly presenting specific stiffness or torsion characteristics.

It is noted that the multiple devices may be coaxial, or may be independently inserted and advanced through a common master device lumen, or yet at least a subset of the set of multiple devices may be advanced each separately in separate master device lumen; or selected device combinations may be advanced through selected master device lumens.

As is known in the art, when RF ablation electrodes, or other types of ablation electrodes are not maintained in optimal contact with the tissues to be ablated, some larger fraction of the applied energy is dissipated in the blood pool surrounding the electrodes; in such a situation the electrodes can overheat the blood and cause coagulum to form on nearby catheter or device structures, thereby reducing intervention capability and effectiveness. It is thus desirable to inject near the electrodes saline to help in cooling the contact surfaces or electrodes. Cooling with saline solution will improve the ablation efficiency. Accordingly the master device lumen, or a selected set of lumen, may be designed and proximally connected to enable the injection of saline, or the injection of contrast medium, during the intervention. Additionally or alternatively, combinations of saline and contrast medium maybe injected proximally to provide for both increased image contrast in the proximity of the treatment area as well as to maintain favorable tissue and blood environment characteristics for the progress of therapy.

According to the present invention, it is also provided for the independent and simultaneous control of two or more devices that are not inserted through the common master device. For illustration, in situations where a lesion is located nearby a vessel branch, it is desirable to advance the master device through the lesion side of the branch, while a separate imaging device maybe advanced through the other vessel branch to allow for real-time imaging of the lesion treatment. Alternatively, and as above, the imaging and treatment devices are inserted through the same master device, but navigated separately so that the imaging device is located in the other vessel branch in a position favorable for the real-time monitoring and guidance of the lesion treatment in the other vessel branch.

It is often desirable to navigate a guide wire to effect a turn with a very small turn radius. It is also often desirable to provide for additional support in a guide catheter or sheath so allow said device to remain inserted in a vessel branch with a small turn radius when another device is inserted through said device. It is known in the art to provide, in one embodiment, a pre-shaped catheter, guide catheter, or sheath. In an alternate preferred embodiment, it is possible to independently and simultaneously navigate a guide wire or ablation wire and a pre-bent sheath. Such independent navigation capability enables positioning and maintaining the pre-bent sheath in a favorable position with respect to a difficult to reach anatomy area, and navigating the wire through a tight turn to either position the guide write in place for subsequent device advance, or to enable treatment of lesions or tissues distally located with respect to a tight turn vessel branch.

Alternatively it is possible to remotely navigate a catheter inserted through a pre-shaped sheath. It should be noted that for each of the various disclosed catheters or medical devices being inserted within a subject's body, at least a portion of the medical device may include an alloy comprising metals selected from consisting of platinum, cobalt, nickel, platinum-iron, iron oxides, or combinations thereof. The selected metals forming the alloy enables the medical device to achieve a desired response to an applied magnetic field in the range of 0.05 tesla to 5.0 tesla, to permit the medical device to be oriented to align the magnetically responsive portion of the medical device with the direction of the applied magnetic field, to thereby provide for navigation of the device.

In a number of applications it is desirable to navigate a pre-shaped lead (assuming a configuration such as a “J” shape) through a catheter; the lead thus formed being capable of being navigated successfully through tortuous anatomy to the point where contact with the tissue, such as the heart tissue wall, is established.

In yet other applications, it is desirable to advance a shaped wire through a remotely controlled catheter. In such applications the capability of independently and simultaneously controlling both the catheter and the shaped wire enable wire navigation through tortuous vessel anatomy. Independent navigation of the catheter enables optimal positioning of the distal catheter end such that independent advance of the shape wire will enable the wire tip to engage a branch vessel at a sharp angle from the proximal vessel through with the catheter navigated.

Simultaneous and independent computer control of a multiplicity of devices facilitates a number of applications, such as percutaneous coronary intervention (PCI). In PCI it is occasionally desirable to probe at the lesion with a smooth wire.

Currently for IC procedures, a physician stands next to the patient and manually advances the wire through the vasculature. While this may be adequate for simple procedures, for longer procedures it exposes the physician to long periods of harmful X-ray radiation. Specifically, treating Chronic Total Occlusions (CTO), which are frequently long procedures that require precise delivery of energy while advancing the wire through the CTO, the manual procedure may not be optimal. The present application provides various embodiments of a wire guide or support catheter, and an inner wire disposed within the wire guide or support catheter. The wire and support catheter may move independently of each other or may move in tandem. Various controlling mechanisms may be employed to allow for advancing the wire and to provide rapid sawing motion of the wire.

In the course of testing new catheter devices and developing new test methods to quantify how well such devices are driven by an advancer, it was discovered that some catheter shaft constructions could not be driven successfully without an unacceptable amount of catheter slip (or failure to drive). It was determined that current advancers relied on trapping the catheter shaft between two spring loaded, grooved drive idler wheels. Only a few grooves on the wheels may contact the shaft at any one time. It was discovered that if the catheter's outside diameter material was too hard or not thick enough to allow the drive wheel grooves to bite or sink in, slippage could also occur. In some catheter constructions, the concentrated spring pressure on the few contact points were sufficient to crush or flatten the catheter shaft, and subsequently cause the catheter body to slip. Rather than using high gripping forces on a few high pressure points, increasing the degree of contact would be necessary so that compressive forces on the catheter could be reduced to thereby reduce the propensity of the drive to flatten the catheter.

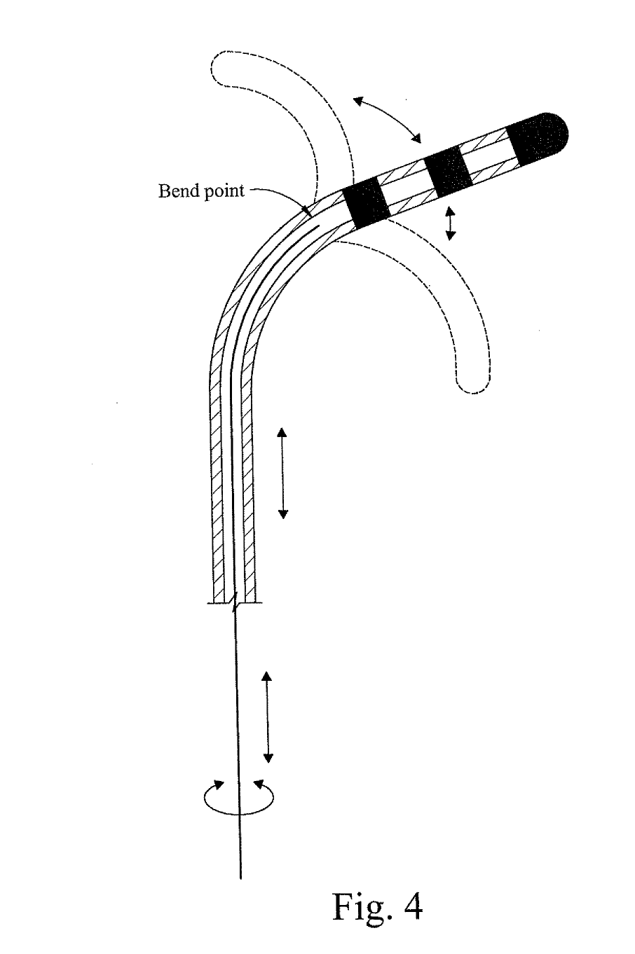

Referring to FIG. 4, a shaped-wire 302 is shown inside of a magnetic Electrophysiology Catheter (EP) catheter 310 that may be advanced by a dual catheter advancing system (CAS). The distal end of the shaped wire 302 may be advanced within an interior lumen of the EP catheter, and provides a movable bend point as the wire moves through the inner lumen of the catheter. The section of the EP catheter distal to the shaped wire is more flexible without the support of the shaped-wire, and is free to deflect through the application of a magnetic field. The Catheter Advancing System may linearly move the EP catheter and wire together, or move the shaped wire/EP catheter relative to each other. The Catheter Advancing System may also rotate the shaped wire within the catheter.

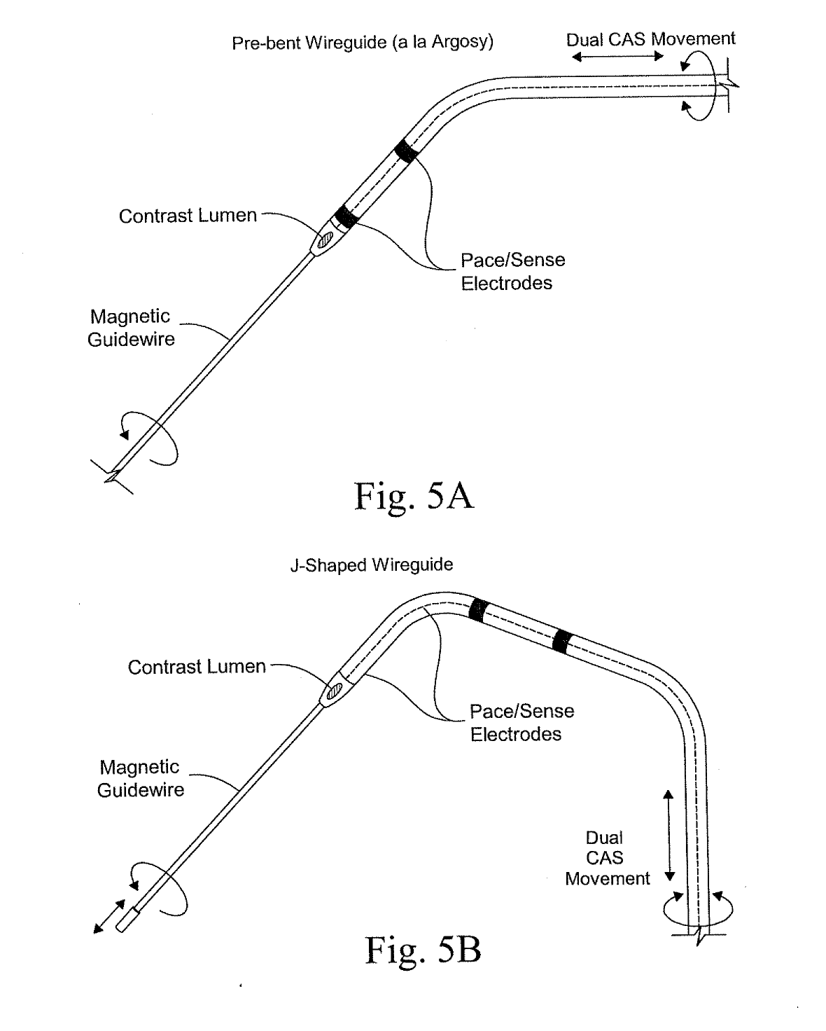

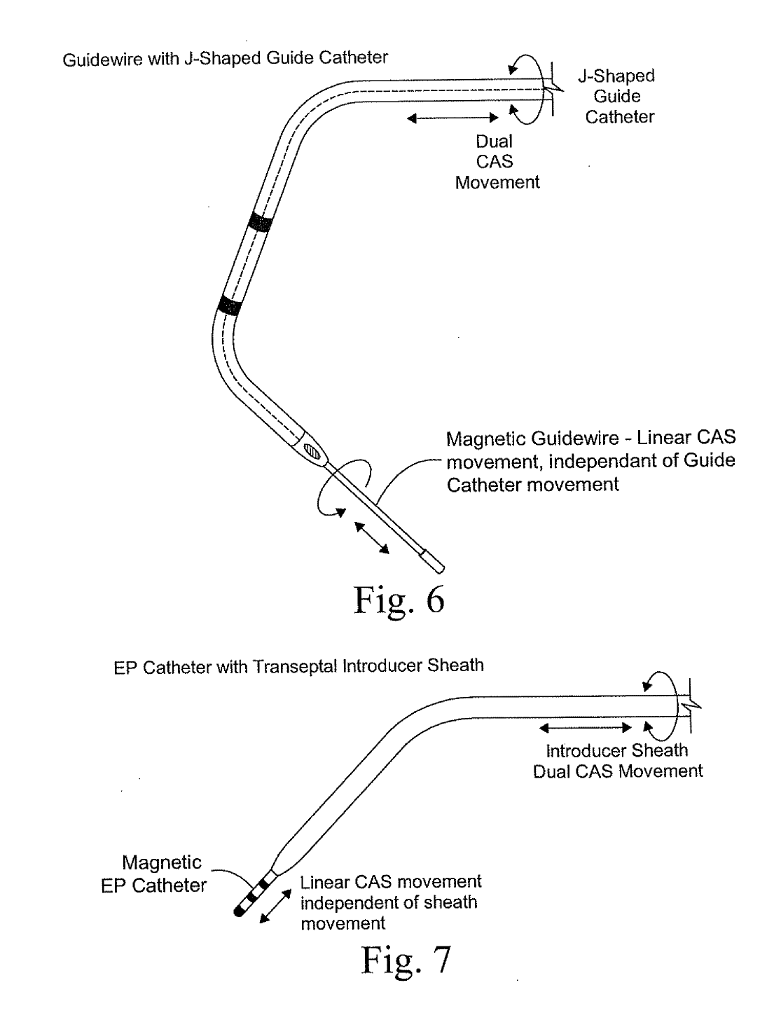

FIG. 5A shows a pre-bent wire-guide 402 having a pre-determined bend angle near the distal end portion 404. The distal end portion 404 further includes pacing electrodes 406 capable of sensing electrical activity. The catheter 410 may include a lumen 412 for delivery of a contrast agent. FIG. 5B shows an alternate configuration of a pre-bent J-shaped wire-guide 502 with pacing electrodes 506 capable of sensing electrical activity. The catheter 510 also includes a lumen 512 for delivery of a contrast agent. FIG. 6 illustrates the CAS movement of the J-shaped wire-guide shown in FIG. 5. FIG. 7 shows an EP catheter 702 disposed within a sheath 710. The catheter 702 has a plurality of magnetically responsive elements 708 disposed on the distal end portion of the catheter 702, and is configured to be moved independent of the sheath 710, via a GAS (not shown).

In addition to the above disclosed features, the bent-tip guide-wires may also be configured for electrically sensing the axial orientation of the bent-tip. The wire preferably contains an index (electronic, mechanical or visual), such as a stripe extending the length of the wire that can be electronically, visually or physically sensed. This sensing can provide feedback to a controller such as a computer, of the actual axial rotational position of the guide-wire. The guide-wire or catheter may further include linear position indicators that may be read or sensed by a sensor that is independent of an advancement/retraction mechanism, which allows for determining the exact linear position of the guide-wire or catheter independent of any possible slippage in the drive mechanism. Moreover, the medical device or catheter may also include an embedded RFID tag, where an RFID reader can detect the type of medical device in use (including information pertaining to the device's length, pre-bent shape, etc.). This information can be communicated to a controller or computer that utilizes the information for control of advancement of the device, where the activation of select advancement mechanisms could be programmed for the particular device.

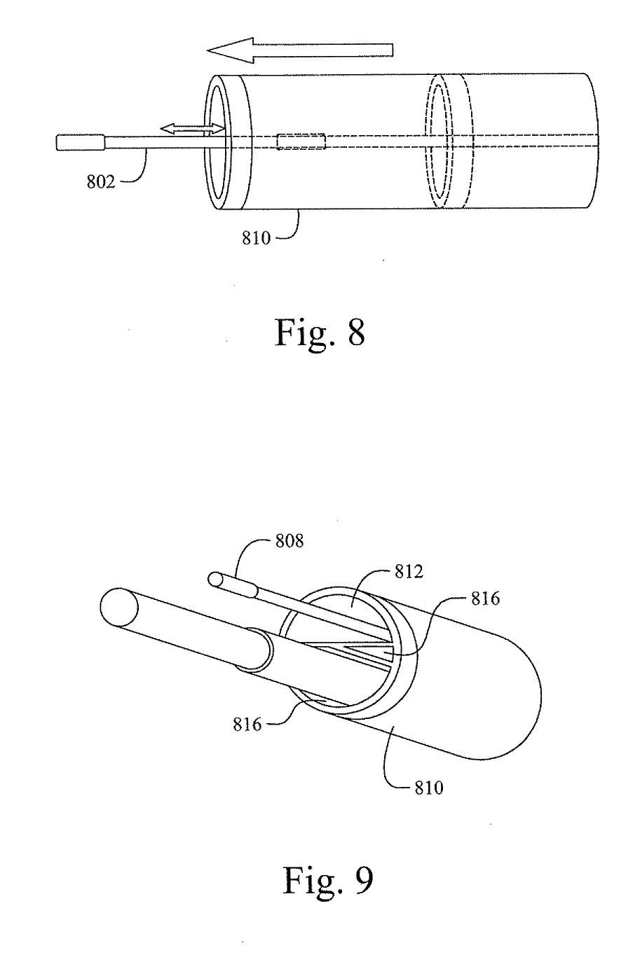

Referring to FIG. 8, a RF wire 802 disposed within a wire-guide 810 is shown. The wire guide 810 is preferably flexible so that it can follow the RF wire with a minimum profile. The wire guide and RF wire 802 together should provide enough support so that the guide can follow the wire inside a lesion. The wire guide 810 and RF wire are configured to move relative to each other, via the Catheter Advancing System. The Catheter Advancing System is configured to provide a unique mode of wire advancement, involving a rapid and small sawing motion of the wire. The sawing frequency could be 10 movements per second, where the back and forth movement is in the range of 0.5 to 1.0 millimeters. When the wire tip is making a turn inside a lesion (with the application of magnetic torque to turn the tip and RF energy for ablation), the sawing motion is critical to making the turn and keeps the tip of the RF wire 802 free of debris.

The wire guide 810 may have at least two lumens, one lumen 812 for the wire 802 and a second lumen 814 for delivery or injection of a contrast agent. The wire guide 810 may further include an additional lumen 816 for receiving an imaging catheter. The imaging catheters movement can be controlled independently as well as in tandem with the wire guide or support catheter, via the CAS. The wire 802 preferably includes one or more magnetically responsive members 808, so as to be magnetically navigable. The CAS is configured to move the wire guide or support catheter and the magnetically navigable wire either independently or together as an assembly.

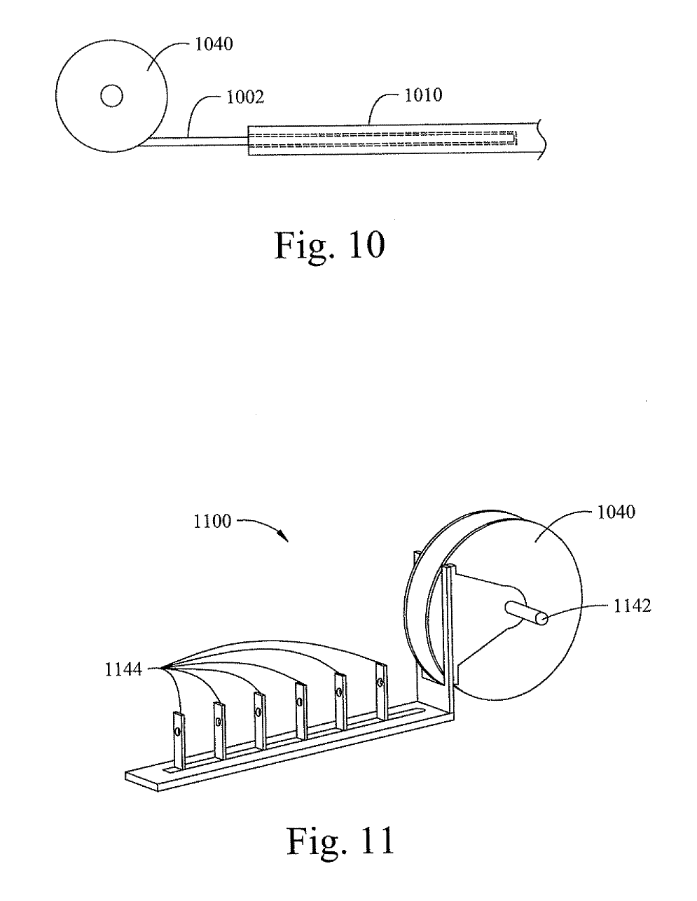

Referring to FIG. 10, one embodiment of a Catheter Advancing System for a core guide-wire 1002 comprises a reel 1040 on which the core guide-wire 1002 may be coiled. A drive (such as a motor) may rotate the reel 1040, which translates rotation into motion of the guide-wire 1002 and/or catheter. The wire guide or support catheter 1010 may include a lumen therein and a stop within the lumen, which stops the guide wire 1002 may engage for advancing the wire guide or catheter 1010.

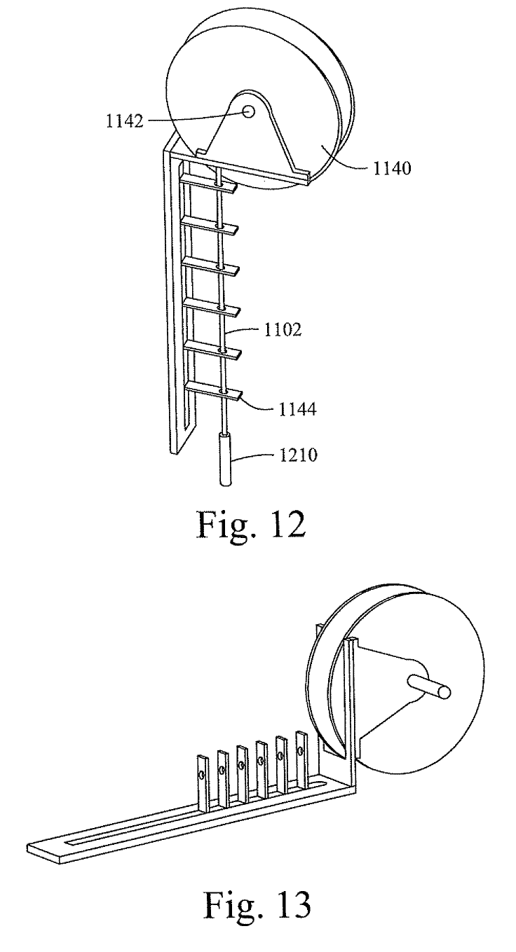

Referring to FIG. 11, a core guide-wire drive mechanism 1100 is shown with a reel 1140 on an axle 1142, about which the reel 1140 is rotated to feed a core guide-wire 1102 through a plurality of guides 1144 and into a device such as a wire-guide or support catheter. As shown in FIG. 12, the core guide-wire 1102 is fed through a plurality of guides 12 and into a catheter device 1210, in which the core guide wire 1102 is locked in place or attached to the device. The motion of the core guide wire 1102 may then be translated to the device. The guides 1144 may be collapsible for allow for additional linear travel of the device, as shown in FIG. 13. It should be noted that while a drive such as a motor may be employed for driving the reel 1140 to advance the guide wire, other linear advancing mechanisms may be utilized for advancing the guide wire from the reel, where such mechanisms can be employed in place of or in combination with a drive for rotating the reel.

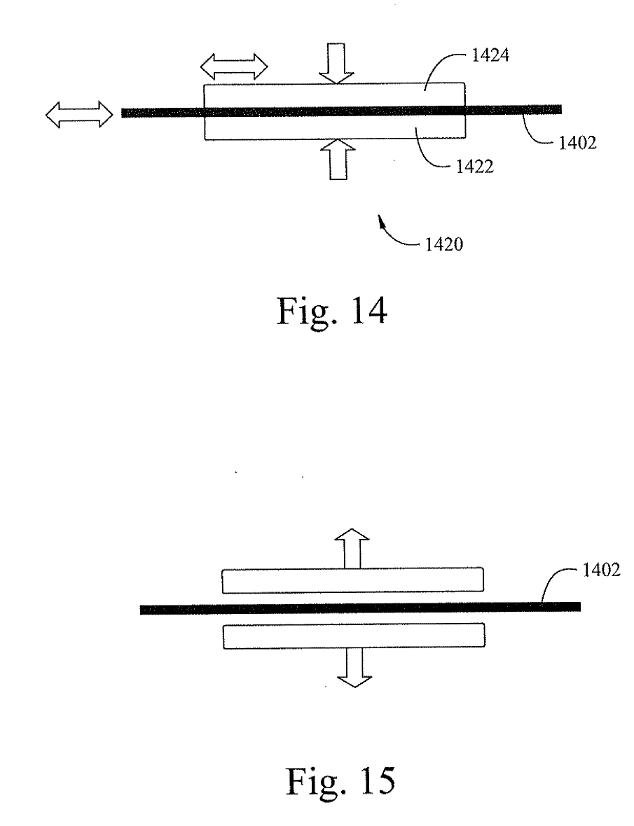

Referring to FIGS. 14 and 15, a conceptual device is shown for linear advancement of a guide-wire or catheter 1402 (or co-axial arrangement of a guide-wire and catheter). The device comprises a linear advancer, which has a mechanism 1420 with two surfaces 1422 and 1424 that contacts the device over an extended surface area, for gently clamping the wire or shaft 1402 between the two surfaces. The device preferably engages opposing sides of the medical device's outer surface over a longitudinal length of at least 0.5 mm, such that the apparatus clamps against the device over an extended length to provide improved surface contact with the medical device over conventional roller guides

Once clamped, movement of the mechanism 1420 provides for movement of the guide-wire or catheter 1402. The two surfaces 1422 and 1424 may be separated from the guide-wire 1402 as shown in FIG. 15. This is unique from advancers that use a set of roller pinch wheels, which put a maximum amount of force at a point contact on the device and causes deformations that may be temporary or possibly permanent. In accordance with the above, various mechanisms for linearly advancing a core or guide-wire are provided.

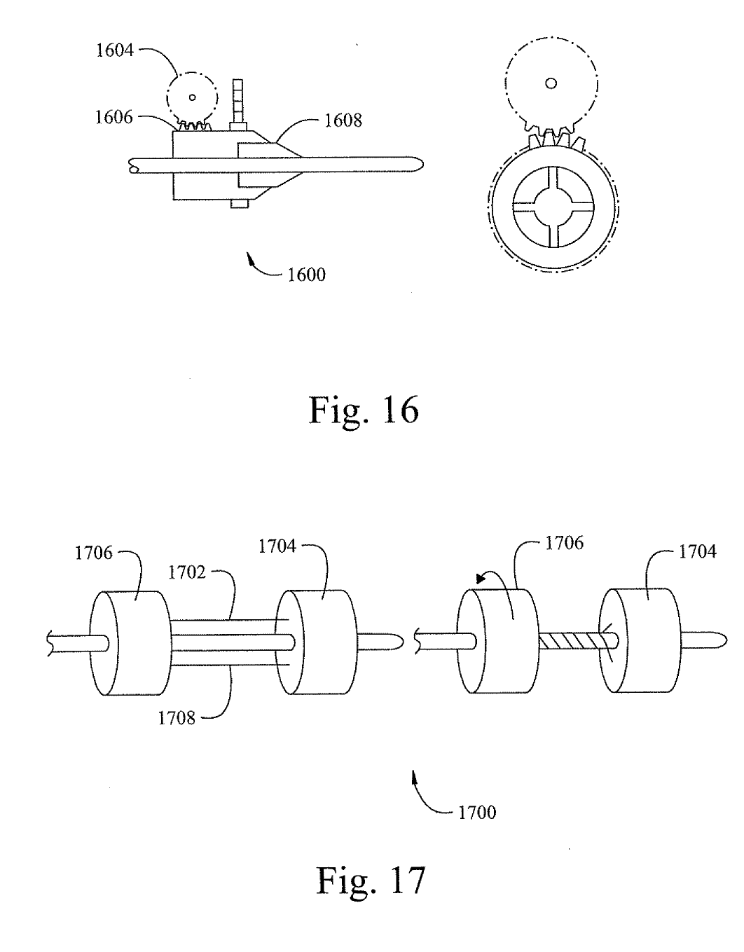

Referring to FIG. 16, one example of a linear advancing mechanism is shown. The mechanism 1600 comprises a rotation wheel 1604 for rotating a collet clamp 1606 having a collet 1608 configured to close around the diameter of a guide wire or medical device. The collet device may be pneumatically actuated, for example, to close against the diameter of the device. Once the collet 1608 is closed, the collet clamp 1606 may be rotated by the rotation wheel, to thereby rotate the guide-wire 1602. A linear wheel is also provided for linearly moving the collet clamp 1606, to thereby linearly advance or retract the medical device. This embodiment may be employed to capture a device and rotate the device or move it longitudinally. Such a mechanism may be advantageously utilized to provide for rapidly advancing and retracting a wire to provide a rapid sawing motion.

Referring to FIG. 17, a braided wire mechanism 1700 is shown that may be used to circumferentially collapse around the diameter of a device. The mechanism comprises a fixed guide 1704 (relative to the medical device or guide wire) and a rotating guide 1706. The mechanism further includes a pair of wires 1708 extending between the fixed guide 1704 and a rotating guide 1706. When the rotating guide 1706 is rotated relative to the fixed guide 1704, the wires wrap around and collapse on the outside diameter of the medical device or guide-wire, for the purpose of capturing the outside diameter. Once the coiled wire has captured the device, continued rotation of the rotating guide will translate into rotation of the device. Similarly, linear movement of the guides will translate into linear displacement of the captured medical device.

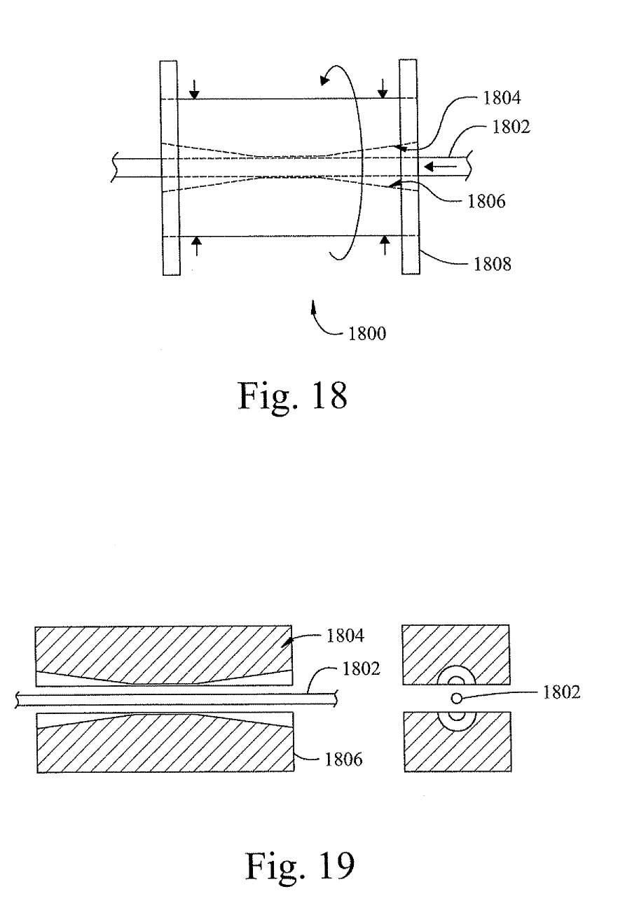

Referring to FIG. 18, a die clamp mechanism 1800 is utilized to clamp on a guide wire 1802, for use in either rotating or shuttling the guide wire 1802 within a catheter, for example. The mechanism includes a first jaw half 1804 and a second jaw half 1806 of a die clamp 1808, which each include a cross-section (as shown in FIG. 19 for example) that is configured for closing on an outside diameter of a guide-wire or medical device. Rotation of the device will occur when the jaws are closed and the die jaws rotate together.

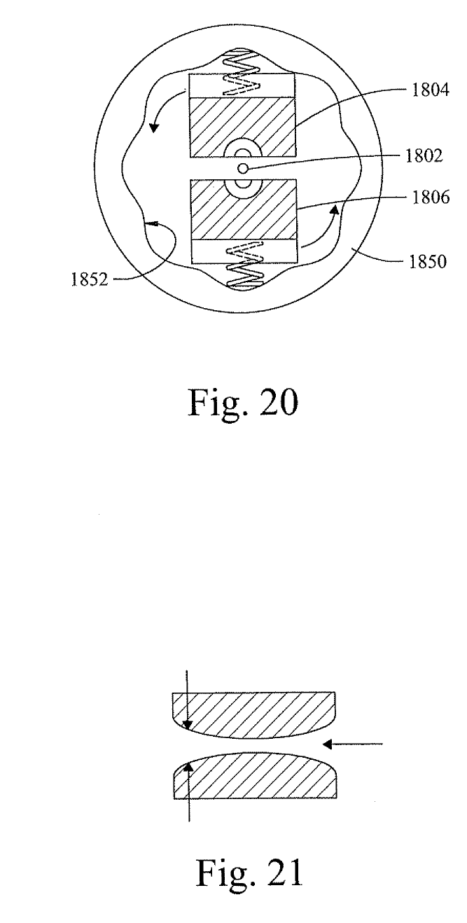

Referring to FIG. 20, rotation of the die jaws may cause the die jaws to be opened and closed by the action of a cammed surface, for example. As shown in FIG. 20, the die halves 1804 and 1806 are positioned around a wire 1802 relative to a member 1850 having an internally cammed surface 1852. The rotation of the die 1808 relative to the cammed surface results in the sequential closing of the dies caused by the cam offset. As the die rotates within the mechanism, a spring-loaded hammer follows the surface of the internal cam to move the jaw halves 1804 and 1806 to an open and closed position. It should be noted that while the die halves 1804 and 1806 may be rotated relative to the stationary cam surface 1852, the die halves may alternatively be stationary while the cammed surface is rotated.

Referring to FIG. 21, an alternate cross-section of the jaws is shown. The particular curved contour of the die jaws permits the rotation relative to the cam surface to initially close the jaws at only the portion indicated by the arrows, such that one end portion of each jaw will capture the outside diameter of the guide-wire or medical device. Further rotation relative to the cammed surface will cause the point of closure of the jaws to move from one end of the jaw to the other. This will have the effect of pushing or propelling the captured portion of the wire, to thereby linearly displace the wire. Such movement is unlike the advancers that use a set of roller pinch wheels, the rotation of which imparts a tangential force at a point contact on the device.

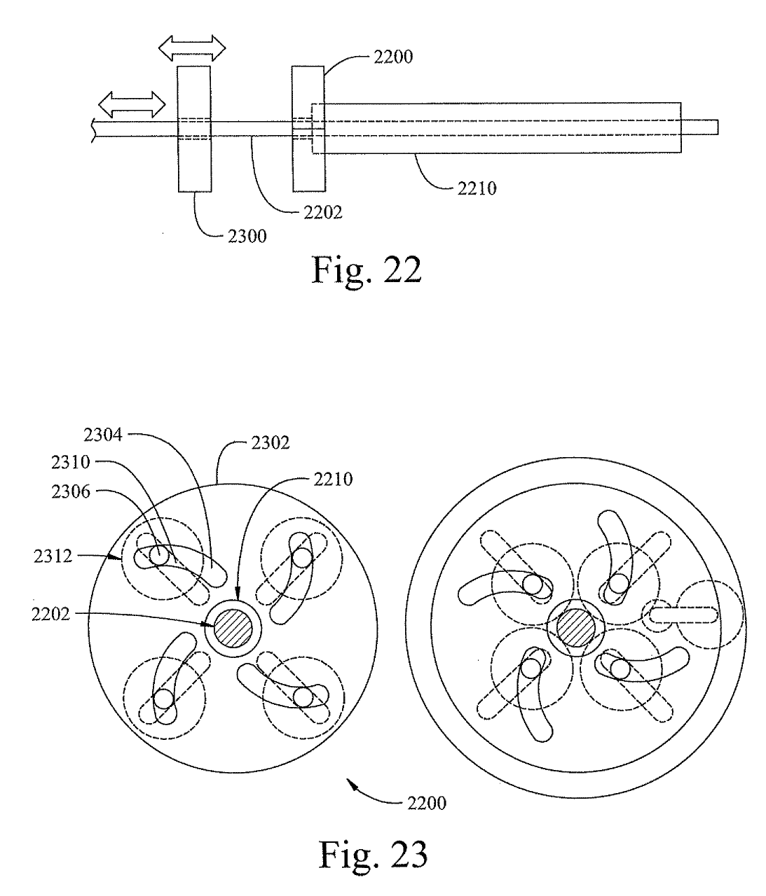

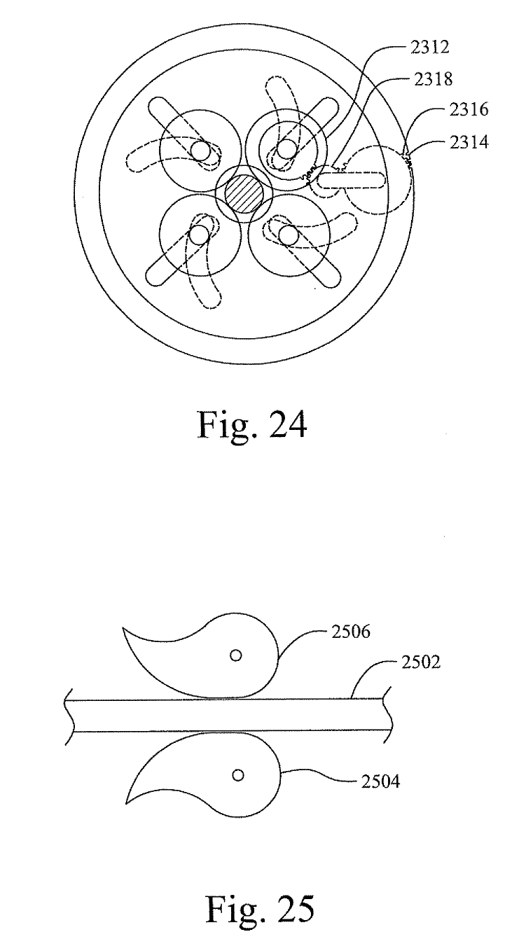

Referring to FIGS. 22 through 24, a slide mechanism 2200 and rotating clamp mechanism 2300 are shown. The slide mechanism comprises a 2-piece clamp that may be used to clamp against a catheter 2210 and to shuttle the catheter 2210 and or guide wire 2202. The rotating clamp 2300 and slide mechanism are utilized to clamp on a guide wire 2202, for use in either rotating or shuttling the guide wire 2202 within the catheter, for example. The mechanism preferably comprises a variable clutch disk 2302 that is used to move the drive wheels onto the outside diameter of a wire or catheter. The clutch disk 2302 includes a plurality of arcuate slots 2304, where the rotation of the disk causes a drive wheel axle 2306 received within the arcuate slot to be displaced along a straight slot 2310. A plurality of drive wheels 2312 are slidably positioned relative to a plurality of straight slots 2310, such that the drive wheels 2312 may slide within the straight slots to accommodate a variable diameter wire concentrically positioned between the drive wheels 2312. By rotating the clutch disk 2302, the drive wheels may be slid inwardly along the straight slots to a position of engagement against a guide-wire or catheter. Once the wheels are in contact with the outside diameter of the device, a force is translated back through a clutch to result in rotation of the drive wheels 2312. Rotation of the drive wheels 2312 is translated through an internal gear 2314 to a couple gear 2316 to a float gear 2318 that is used to drive the drive wheel 2312. The clamp force regulated by the clutch will result in a set amount of compression on the guide-wire or catheter device. Each drive wheel may be rotated by the same mechanism (such as a motor) that the clutch disc uses. This is unique from advancers that use a set of roller pinch wheels, which put a maximum amount of force at a point contact on the device and causes deformations.

Referring to FIG. 25, another embodiment of an advancement mechanism is shown with an opposing cam mechanism. The mechanism 2500 includes a pair of cams 2504 and 2506 that are spring loaded to rotate closed against a guide-wire or medical device 2502. As the rotation axis of the cam moves away from the device, the cam translates rotational motion to linear displacement of the captured or gripped portion of the guide-wire or medical device. At a distance from the device, the cam resets by temporarily disengaging from the device and reloading the spring force to reset the cam on a new location. Thus, the cams are configured to iteratively provide linear movement of the wire.

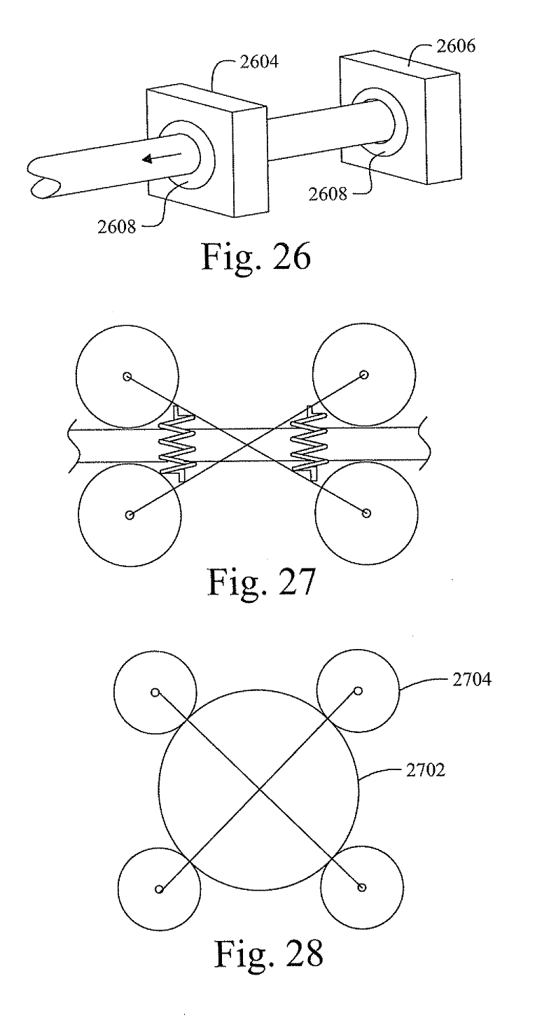

Referring to FIG. 26, another embodiment of an advancement mechanism is shown. The mechanism comprises a pair of blocks 2604 and 2608, each having an inflatable o-ring. In this arrangement, the first block 2604 is inflated to engage the outside diameter of the guide-wire, catheter or medical device, and hold it stationary while the second block 2606 is moved away. The o-ring 2608 of the second block 2606 is then inflated to hold the wire device. Once the second block o-ring is inflated, the first block o-ring is deflated, and the second block 2606 is moved. The deflated o-ring in the first block allows the wire/medical device top slide through while the second block linearly moves the wire/medical device.

Referring to FIG. 27, another embodiment of an advancement mechanism is shown with a cross-drive arrangement of drive wheels 2704. The purpose of the cross drive wheels 2704 is to impart linear motion to the guide wire or catheter 2702. A spring force is used for biasing the drive wheels to engage the medical device.

Referring to FIG. 28, another embodiment of cross-drive arrangement of drive wheels is shown in a gear and train configuration. In this embodiment, a motor torque is transferred through the linkage of gears to apply a force for drive wheel engagement. A pair of sun gears 2804 to produce rotation in tow drive wheels 2806 while a pair of idle wheels 2808 remain idle.

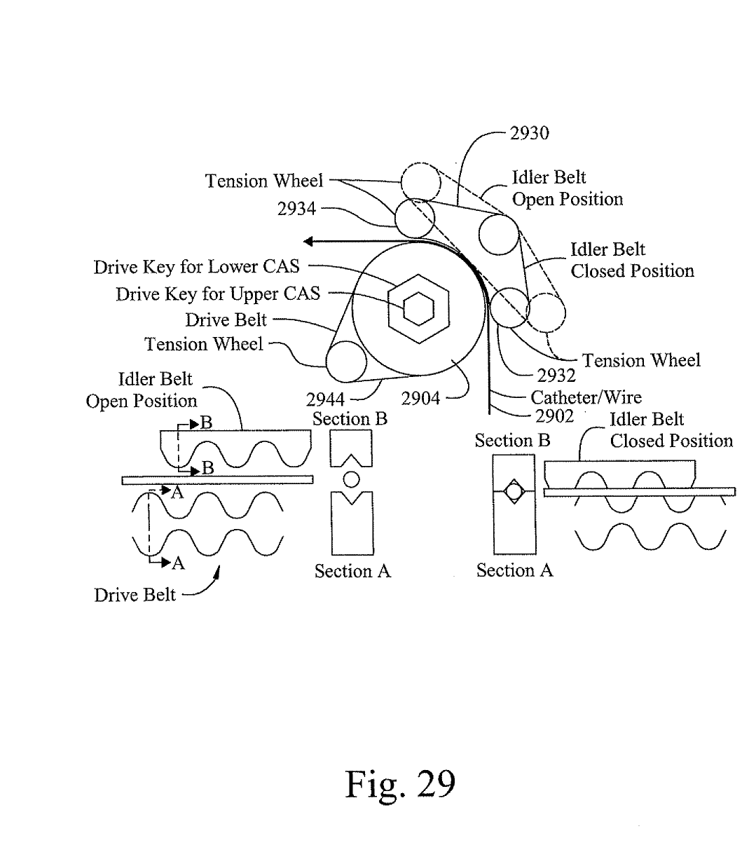

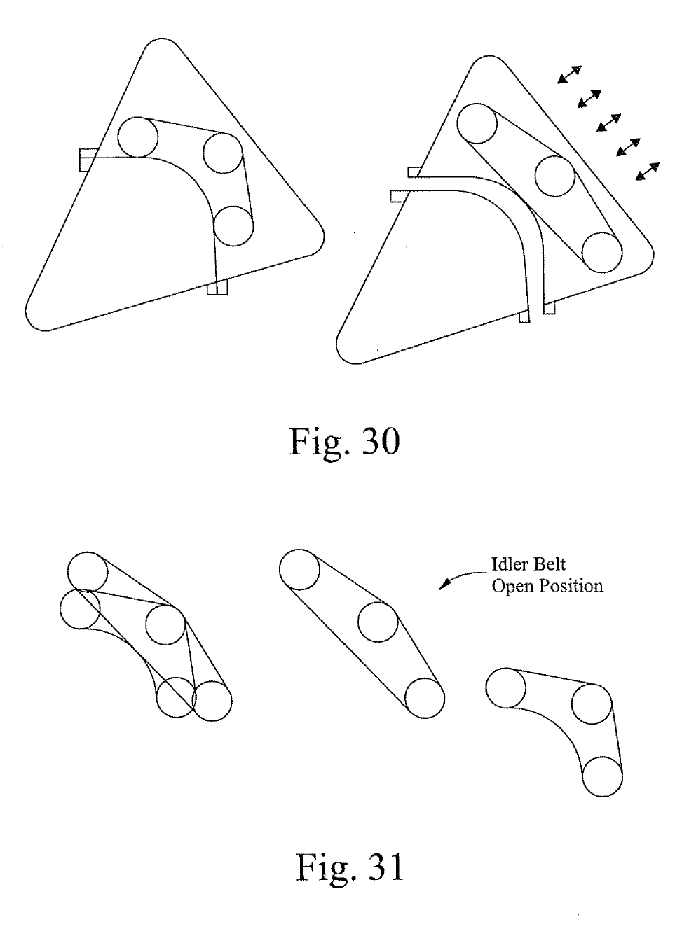

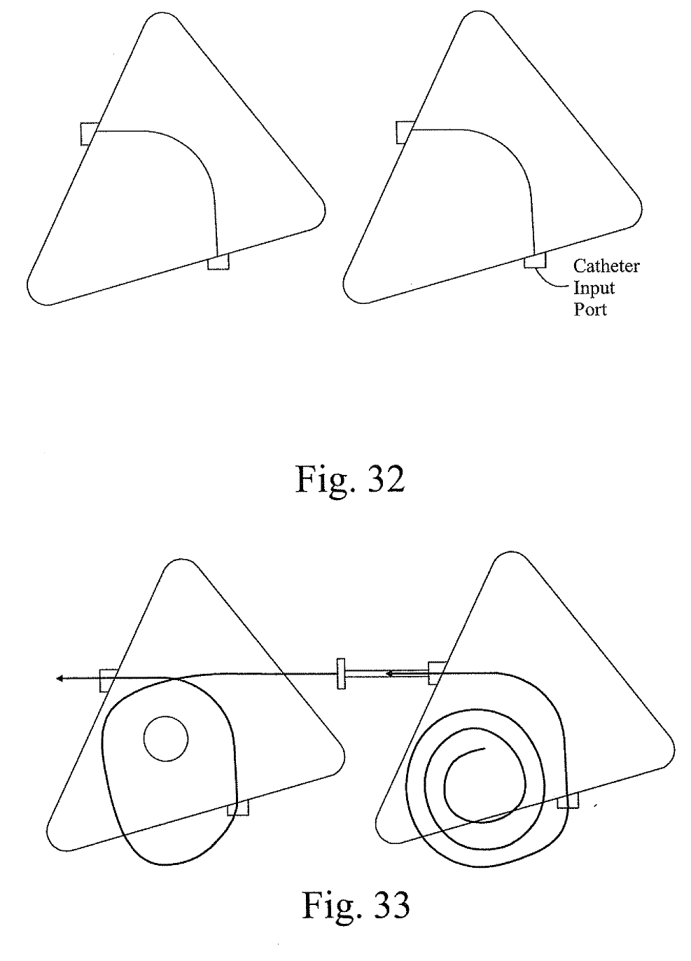

Referring to FIGS. 29-33, a caterpillar drive that involves using drive belts or tracks to produce longitudinal motion of a guide-wire, catheter or medical device. The mechanism comprises an idler belt 2930 wrapped around a pair of movable tension wheels 2932 and 2934, where the engagement of the idler belt 2930 against a guide wire/catheter 2902 positioned against a drive wheel 2940 causes the displacement of the tension wheels 2932 and 2934 for positioning the portion of the idler belt 2930 extending between the tension wheels into substantially full contact with the outer surface of the guide-wire or catheter 2902. A drive belt 2944 is configured to rotate the drive wheel 2940, which rotation translates into linear displacement of the guide-wire or catheter 2902.

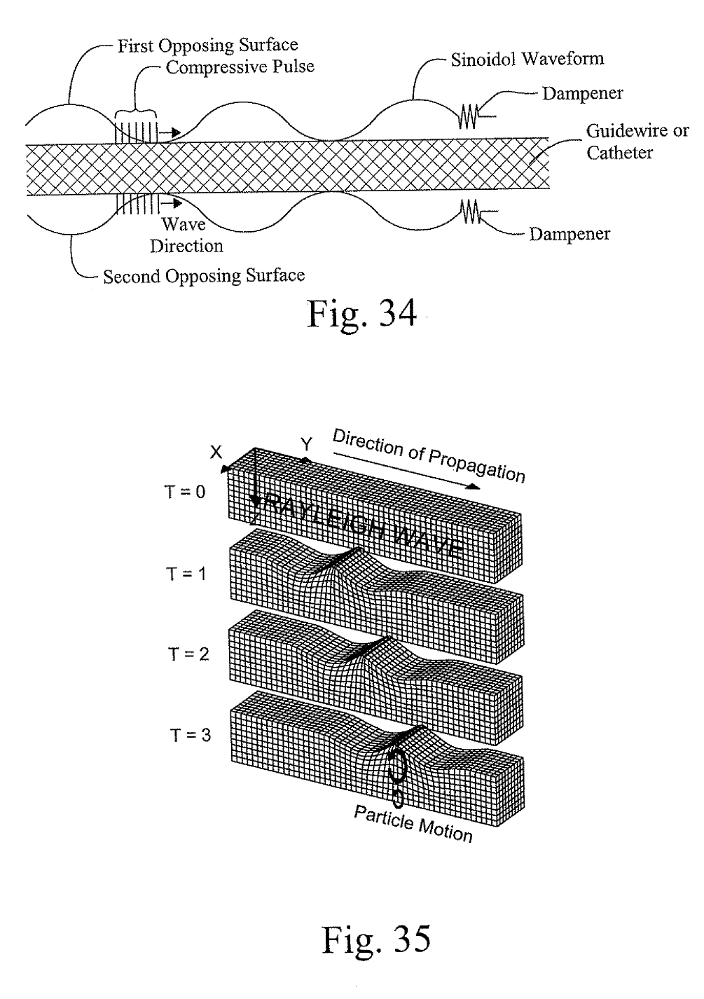

Referring to FIGS. 34 and 35, another embodiment of an advancement mechanism is shown that comprises a compressive wave drive. As shown in FIG. 35, a complex waveform called a Rayleigh Wave is a surface wave that can impart circular motion opposing the direction of wave propagation. Upon establishing two opposing surfaces having the same wave frequency, the resulting motion of the wave can be transferred to a guide-wire or catheter between the crests of the opposing wave forms. The purpose of the wave drive is to translate vibratory and compressive energy into longitudinal motion of a wire-guide or catheter shaft. Wave propagation via a vibratory oscillator will produce a sinusoidal wave form to capture the device, where the compressive wave form will move the device. The sine wave will roll over some length with the compressive wave propagating at the same rate or in pulses at the crest of the sinusoidal wave frequency.

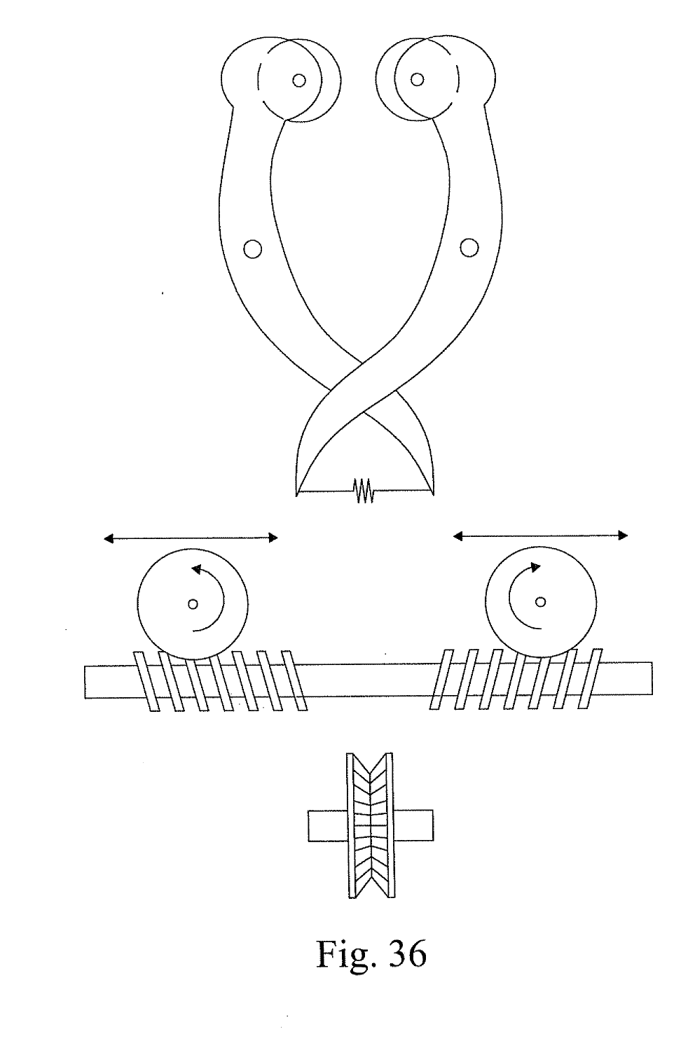

Referring to FIG. 36, another embodiment of an advancement mechanism is shown that comprises a rolling worm gear drive mechanism. The worm drive will allow for multiple drive wheels to be driven. The unique function of the two worm gear shaft is the ability for the distance between the drive wheels to be adjusted based on the size of the device that is positioned between the drive wheels for advancement. The drive gears can roll along the worm gear to a location where both drive wheels engage the device.

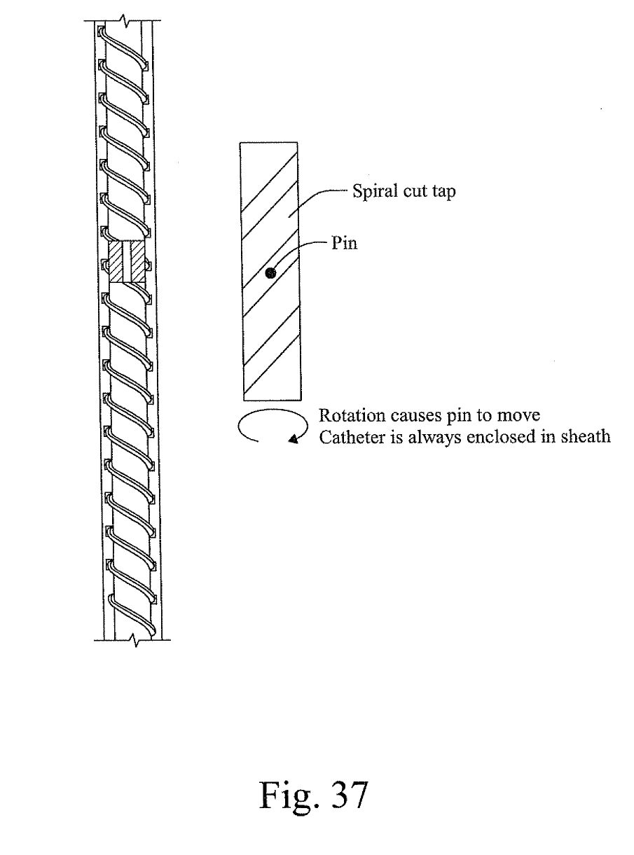

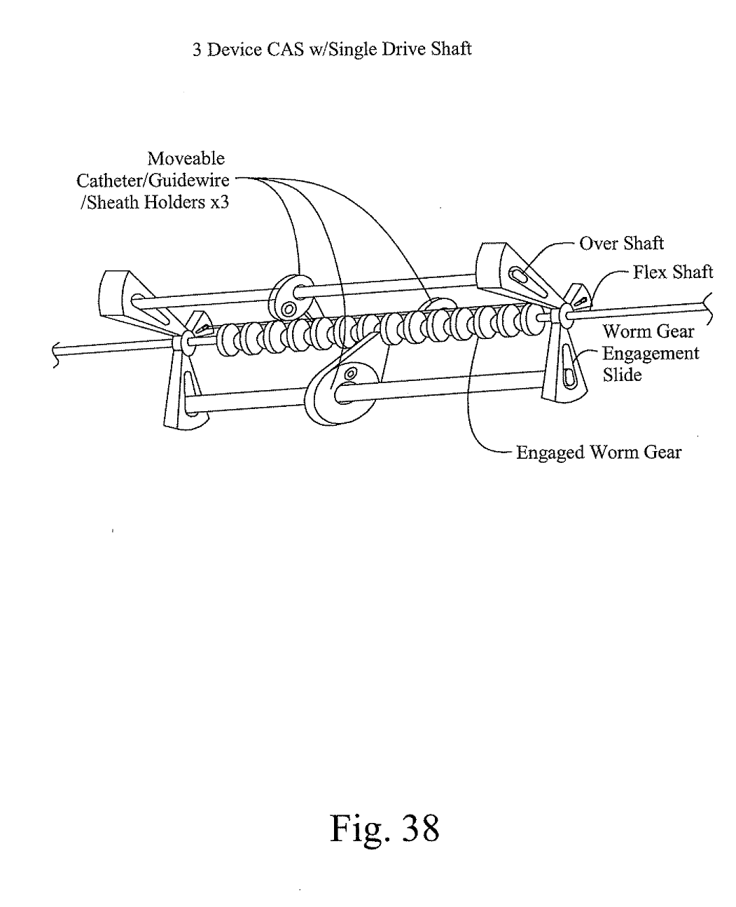

Referring to FIGS. 37-38, another embodiment of an advancement mechanism is shown that comprises a rolling worm gear drive mechanism.

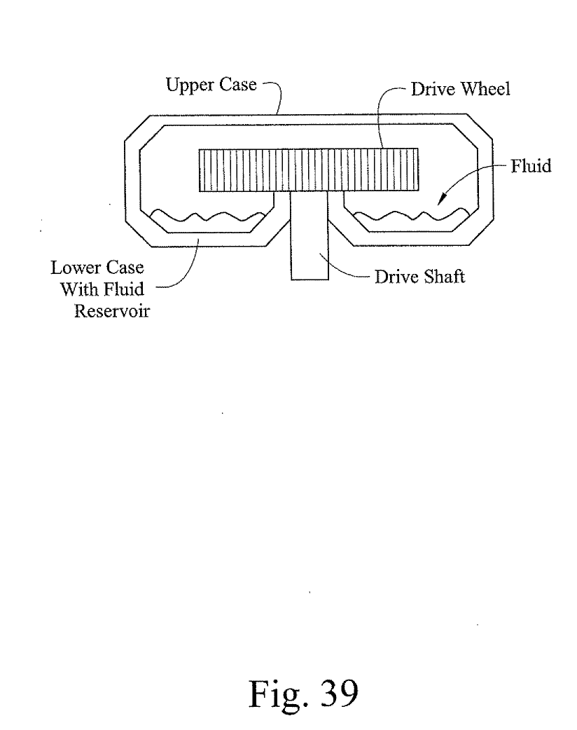

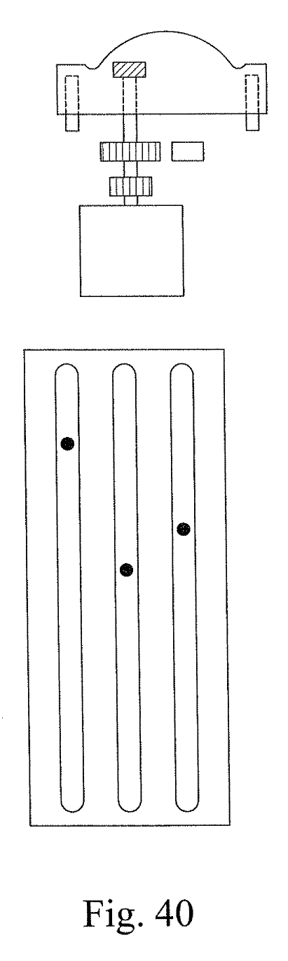

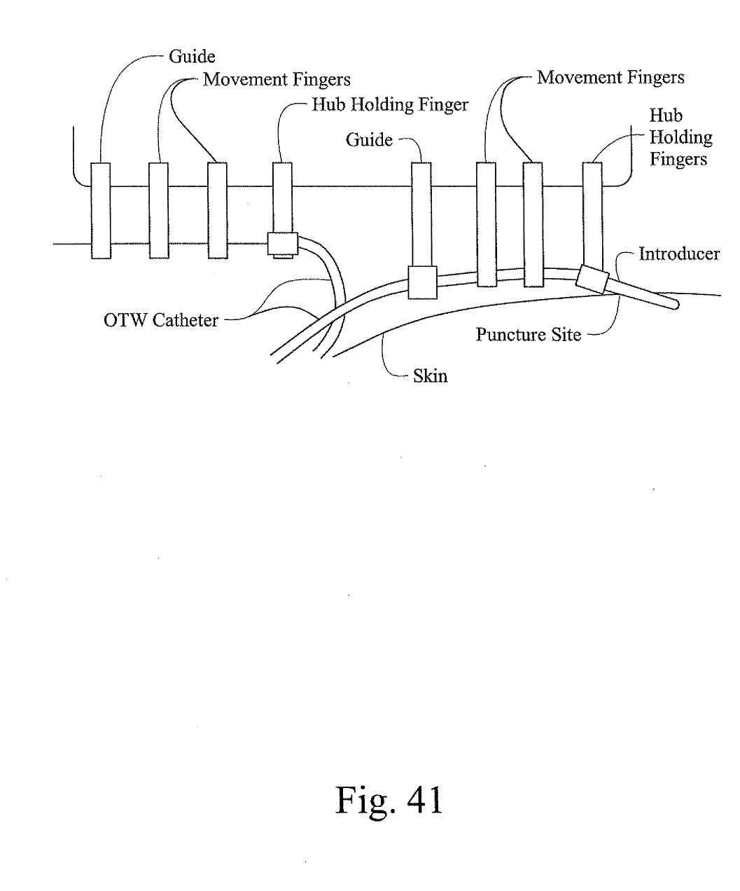

Referring to FIG. 39, another embodiment of an advancement mechanism is shown that comprises a low-melt allow drive, where a low melt alloy propelled by a drive wheel may be used to grab a guide-wire or catheter when in solid form, and flow around the guide-wire or catheter when in liquid form.

The advantages of the above described embodiments and improvements should be readily apparent to one skilled in the art, as to enabling delivery of cells or similar therapeutic agents or particles to a targeted organ or organ surface. Additional design considerations may be incorporated without departing from the spirit and scope of the invention. Accordingly, it is not intended that the invention be limited by the particular embodiments or forms described above, but by the appended claims.

What is claimed is:

1. A method of controlling an apparatus to advance a medical device within a subject body, the method comprising:
inserting a medical sleeve device comprising a hollow lumen in the subject body;
Inserting a medical device within the hollow lumen of the medical sleeve device;
actuating an apparatus that is configured to engage, upon actuation, the outer surface of the medical device at more than two points about the circumference of the medical device,
orienting the medical device distal tip to guide the medical device towards the vicinity of the organ wall; and
moving the apparatus to advance the medical device within the subject towards contact with the organ wall of the subject.

2. The method of claim 1, wherein the apparatus is configured to collapse around the outside diameter of the medical device so as to capture the medical device.

3. The method of claim 1, wherein the apparatus is configured to engage the outer surface of the medical device over a longitudinal length of at least 0.5 mm, such that the apparatus clamps against the device over an extended length to provide improved surface contact with the medical device over conventional roller guides.

4. The method of claim 1, wherein movement of the apparatus establishes linear displacement of the medical device to advance the medical device into the subject's body.

5. The method of claim 1, wherein a portion of the apparatus is configured to rotate and thereby establish rotation of the medical device within the subject's body.

6. The method of claim 1, wherein the advancing apparatus is configured to move the medical sleeve device or the medical device within the sleeve's hollow lumen, either independently or together as an assembly.

7. The method of claim 1, further comprising the step of deploying a medical implant through the hollow lumen in the medical device to the organ wall, to permit delivery of the medical implant to the organ wall in the subject's body.

8. The method of claim 1 wherein the applied magnetic field comprises a field that is essentially uniform across the target area including the organ wall.

9. A method of delivering therapeutic substances to a target area in a subject's body, the method comprising:
inserting a medical sleeve device comprising a hollow lumen in the subject body;
Inserting a medical device within the hollow lumen of the medical sleeve device;
actuating an apparatus that is configured to engage on opposing sides of the medical device's outer surface over a longitudinal length of at least 0.5 mm, to clamp against a longitudinal portion of the medical device;
orienting the medical device distal tip to navigate the medical device towards the target area of the subject's body; and
moving the apparatus to advance the medical device towards the target area.

10. The method of claim 9, wherein the apparatus is configured to collapse around the outside diameter of the medical device so as to capture the medical device.

11. The method of claim 9, wherein the apparatus is configured to engage the outer surface of the medical device over a longitudinal length of at least 0.5 mm, such that the apparatus clamps against the device over an extended length to provide improved surface contact with the medical device over conventional roller guides.

12. The method of claim 9, wherein movement of the apparatus establishes linear displacement of the medical device to advance the medical device into the subject's body.

13. The method of claim 12, wherein a portion of the apparatus is configured to rotate and thereby establish rotation of the medical device within the subject's body.

14. The method of claim 9, wherein the advancing apparatus is configured to move the medical sleeve device or the medical device within the sleeve's hollow lumen, either independently or together as an assembly.

15. An apparatus for advancing a medical device for delivering therapeutic substances to a target area in a subject's body, the apparatus comprising:
one or more engaging portions configured to engage the outer surface of a medical device at more than two points about the circumference of the medical device, such that the one or more engaging portions engage opposing sides of the medical device's outer surface over a longitudinal length of at least 0.5 mm, to clamp against a longitudinal portion of the medical device; and
an actuation means for causing the one or more engaging portions to clamp against the outer surface of a medical device; and
a rotation means for establishing rotation of a portion of the apparatus engaged with the medical device, for effecting rotation of the medical device.

16. The apparatus of claim 15, wherein the apparatus is configured to collapse around the outside diameter of the medical device so as to capture the medical device.

17. The apparatus of claim 15, wherein the apparatus is configured to engage the outer surface of the medical device over a longitudinal length of at least 0.5 mm, such that the apparatus clamps against the device over an extended length to provide improved surface contact with the medical device over conventional roller guides.

18. The apparatus of claim 15, wherein movement of the apparatus establishes linear displacement of the medical device to advance the medical device into the subject's body.

19. The apparatus of claim 15, wherein the apparatus is configured to move the medical device or the medical guide device within the hollow lumen, either independently or together as an assembly.

20. The apparatus of claim 15, further comprising engaging means configured to engage the outer surface of a medical sleeve having a lumen in which the medical device is inserted, wherein the apparatus is configured to move the medical sleeve or the medical device within the sleeve's hollow lumen, either independently or together as an assembly.

21. The apparatus of claim 20 wherein at least a portion of the medical device includes an alloy comprising metals selected from consisting of platinum, cobalt, nickel, platinum-iron, iron oxides, or combinations thereof, wherein the alloy achieves a desired response to an applied magnetic field in the range of 0.05 tesla to 5.0 tesla.
